(19) United States
(12) Patent Application Publication
(10) Pub. No.: US 2012/0330132 A1
(43) Pub. Date: Dec. 27, 2012

(54) Device for the Delineation of Cardiovascular or Other Anatomical Structures

(76) Inventors: Paul Sorajja, Rochester, MN (US); Charanjit S. Rihal, Rochester, MN (US)

(21) Appl. No.: 13/514,039

(22) PCT Filed: Dec. 3, 2010
(86) PCT No.: PCT/US10/58954

§ 371 Date: Sep. 11, 2012

Related U.S. Application Data
(60) Provisional application No. 61/267,344, filed on Dec. 7, 2009.

(51) Int. Cl. A61B 6/00; A61B 5/055

(52) U.S. Cl. 600/420; 600/435; 600/425

(57) ABSTRACT

A method and device for delineating structures includes a catheter for insertion at the structure, an expandable component operatively having proximal, distal and intermediate segments shiftable between a collapsed state and an expanded state, with the intermediate segment shiftable independently of the proximal and distal segments. A marking medium is carried by a portion of the intermediate segment, and the marking medium detectable using a selected imaging device. The marking medium is arranged to be transferable from the intermediate segment to portions of the cardiovascular structure upon shifting the intermediate segment to the expanded position with the intermediate segment in the desired position.

### FIELD OF THE DISCLOSURE

The present disclosure generally relates to medical devices and, more particularly, to a system and method for delineating the location and/or orientation of an anatomical structure in preparation for treatment of the structure.

### BACKGROUND

Patients with heart disease suffer from a wide variety of intrinsic structural defects and abnormalities of the great vessels. Examples of these diseases include, for example, valvular stenosis and regurgitation, congenital defects (such as aortic coarctation, patent ductus arteriosus, atrial septal defect, ventricular septal defect, patent foramen ovale), and iatrogenic disorders (pulmonary vein stenosis due to radiofrequency ablation, pen-prosthetic valvular regurgitation). With modern advancements in endovascular catheterization, there has been increased development of percutaneous therapies for the foregoing conditions or defects, and for other vascular or anatomic lesions. Using catheterization techniques, various medical devices are placed at the site of the lesion, thereby enabling the operator to deliver the appropriate therapy to the patient (e.g., balloon dilatation, defect closure, valve replacement, etc.). These percutaneous therapies typically require adjunctive imaging with fluoroscopy and/or echocardiography to be performed successfully.

Nonetheless, in many applications there may be significant limitations with current imaging techniques that inhibit accurate and precise placement of medical devices within structural defects and vascular lesions. In general, conventional imaging techniques provide detailed two-dimensional renderings, but structural defects and vascular lesions are three-dimensional in shape, size, and orientation. The operators therefore are required to mentally reconstruct the target defects and lesions from various non-simultaneous, two-dimensional imaging captures. Temporary devices (e.g. sizing balloons, balloons used for angioplasty of stenotic lesion) delineate structural defects and vascular lesions and can be seen during replays of cineangiographic images. However, during fluoroscopic positioning of the permanent medical device, the shape, size, and orientation of the structural lesion cannot be visualized accurately with, for example, current x-ray imaging techniques. These limitations inhibit the operator's ability to choose the correct device size, the optimum location for the device, and the most appropriate orientation of the permanent device.

### SUMMARY

One aspect of the present disclosure provides a device for delineating a cardiovascular structure and comprising a catheter sized for insertion to a position adjacent the cardiovascular structure, an expandable component operatively coupled to a distal end of the catheter, with the expandable component including a proximal segment, a distal segment, and an intermediate segment, and with the expandable component sized to permit the proximal and distal segments to be disposed on opposite sides of the cardiovascular structure with the intermediate segment in a desired position adjacent the cardiovascular structure. Each of the proximal, distal and intermediate segments are shiftable between a collapsed state and an expanded state, with the intermediate segment shiftable independently of the proximal and distal segments. A marking medium is carried by a portion of the intermediate segment, with the marking medium detectable using a selected imaging device. The marking medium is arranged to be transferable from the intermediate segment to portions of the cardiovascular structure upon shifting the intermediate segment to the expanded position with the intermediate segment in the desired position.

In further accordance with one or more preferred forms, the expandable component may comprise a memory metal, an expandable foam, or a balloon. The marking medium preferably may comprise a substance detectable using x-ray imaging, ultrasound, or other medical imaging or detection techniques including, for example, optical coherence tomography, ultrasound, magnetic resonance imaging, x-ray imaging, fluorescence imaging, near-infrared fluorescence (NIRF) imaging, or a camera.

The marking medium may include a therapeutic agent carried by the expandable component, and the therapeutic agent and the marking medium may be mixed with one another. The marking medium may be chemically or mechanically adhered to the expandable component, and mechanical attachment may be accomplished by having fold lines in the expandable component when in an initial collapsed state, with the marking medium secured within the fold lines when the intermediate segment is in the initial collapsed state. The marking medium also may be combined with an adherent substance, the adherent substance arranged for differential adherence to different tissues, and may further include means to facilitate deployment of the marking medium, with the means including, for example, at least one of FR energy, ultrasound, magnetic energy, chemical bonding, thermal energy, and fluid pressure. The marking medium may be bioabsorbable.

In further accordance with an exemplary aspect of the invention, a method for delineating an anatomical structure may comprise providing a catheter sized for insertion to a position adjacent the structure, providing an expandable component operatively coupled to a distal end of the catheter, the expandable component including a proximal segment, a distal segment, and an intermediate segment, positioning the expandable component to place the proximal and distal segments on opposite sides of the structure, with the intermediate segment in a desired position adjacent the cardiovascular structure, equipping each of the proximal, distal and intermediate segments to be shiftable between a collapsed state and an expanded state, equipping the intermediate segment to be shiftable independently of the proximal and distal segments, providing at least the intermediate segment with a marking medium carried by a portion of the intermediate segment, the marking medium detectable using a selected imaging device, positioning the device adjacent the anatomical structure, and expanding the segments to transfer the marking medium from the intermediate segment to portions of the anatomical structure.

The method may include deflating the segments after transferring the marking medium to the structure, and may include withdrawing the catheter and the expandable component after deflation, and using an imaging device to ascertain characteristics of the structure, and may further include using the characteristics to position a medical device.

Preferably, the method may include delivering a therapeutic agent through the expandable component, may include mixing the therapeutic agent and the marking medium, may include chemically adhering the marking medium to the expandable component, and may include carrying the marking medium on the intermediate segment of the expandable component by placing the marking medium in fold lines in the expandable component. Preferably, the marking medium may be combined with an adherent substance, the adherent substance arranged for differential adherence to different tissues, and may include providing the marking medium with means facilitate transfer of the marking medium to the structure, wherein the means is selected from a material responsive to at least one of FR energy, ultrasound, magnetic energy, chemical bonding, thermal energy, and fluid pressure.

Preferably, in accordance with one more of the exemplary forms outlined herein, the disclosed device may permit the user to visualize the anatomic structure in real-time. This may off advantages over existing devices or methods, in that existing visualization techniques require reconstruction of previous images (e.g., road map, CT scans, etc.). Simultaneous fluoroscopy using conventional devices and methods cannot visualize enough detail of any of the targeted anatomical structures. Still preferably, the expandable component, such as a balloon or other expandable component, is preferably compliant. Still preferably, the preferred expandable components preferably may conform to the shape of the anatomical structure to be marked, in such a manner that the marking may be accomplished prior to displacing the anatomical structure itself. This may be accomplished by enabling the expandable component, such as a balloon, to be inflated or expanded only partially prior to full inflation or expansion. This may enable the expandable component to fill in, for example, crevices and other areas at the anatomical structure, which may enhance marking of the structure. In applications where the anatomical structure is to be displaced by the expandable component, the marking is thus accomplished prior to full expansion. In a further exemplary aspect, when using a compliant balloon device, the folds of the balloon fill all of the crevices, such as those found around an aortic valve. Consequently, the valve is marked before the valve is disrupted or displaced by full balloon inflation. By enveloping the structure first, the positioning of the permanent device is enhanced and different from the guesswork made by the operator without using one or more of the preferred forms outlined herein.

### BRIEF DESCRIPTION OF THE DRAWINGS

FIG. 1A is an enlarged fragmentary cross-sectional view of a marking device assembled in accordance with the teachings of a first disclosed example of the present invention with an expandable component in the form of a balloon having a marking device positioned across a valve, such as the aortic valve.

FIG. 1B is another cross-sectional view similar to FIG. 1A but illustrating the expandable component in an expanded state.

FIG. 1C is a further cross-sectional view similar to FIGS. 1A and 1B and illustrating the expandable component in a collapsed state after having deposited a marking medium on the leaflets and the surrounding annulus of the valve.

FIG. 2A is an enlarged fragmentary cross-sectional view of a marking device assembled in accordance with the teachings of a second disclosed example of the present invention with an expandable component in the form of a segmented balloon having a marking medium, with the device positioned across a valve.

FIG. 2B is another cross-sectional view similar to FIG. 2A but illustrating the proximal and distal segments of the balloon in expanded states to help position the collapsed middle or intermediate segment adjacent the valve.

FIG. 2C is another cross-sectional view similar to FIGS. 2A and 2B but illustrating the middle segment of the balloon in an expanded state.

FIG. 2D is a further cross-sectional view similar to FIGS. 2A through 2C and illustrating the entire balloon in a collapsed state after having deposited the marking medium on the leaflets and the surrounding annulus of the valve.

FIG. 3A is an enlarged fragmentary cross-sectional view of a marking device assembled in accordance with the teachings of a third disclosed example of the present invention with an expandable segmented Inoue balloon having a marking medium, with the device positioned across a valve.

FIG. 3B is another cross-sectional view similar to FIG. 3A but illustrating the distal segment of the balloon in an expanded state to help position the collapsed middle segment adjacent the valve.

FIG. 3C is another cross-sectional view similar to FIGS. 3A and 3B but illustrating the proximal and middle segment of the balloon in a partially expanded state.

FIG. 3D is a further cross-sectional view similar to FIGS. 3A through 3C and illustrating the balloon in a fully expanded state in a position to deposit the marking medium on the leaflets and the surrounding annulus of the valve.

FIG. 4A is an enlarged view of an exemplary expandable component in the form of a balloon and having the marking medium arranged in a pattern of discrete dots or points and equipped for securement with either barbs (shown in a further enlarged fragmentary view to the right of FIG. 4A) or an adhesive (shown in another further enlarged fragmentary view to the right of FIG. 4A).

FIG. 4B is an enlarged view of another exemplary expandable component also in the form of a balloon and having a marking medium arranged in a circumferential band, with the marking medium treated with an adhesive as shown in a further enlarged fragmentary cross-section to the right of FIG. 4B.

FIG. 4C is an enlarged view of a further exemplary expandable component in the form of a balloon having fold lines went in the collapsed state shown with the marking medium secured within the fold lines when the balloon is in the collapsed state.

FIG. 4D is an enlarged view of still another exemplary expandable component in the form of a double-walled balloon having the marking medium disposed between the balloon walls and ejectable through pores in the outer balloon upon inflation of the inner balloon, with the marking medium shown in a space between the two balloons in the fragmentary cross-sectional view to the left of FIG. 4D.

FIGS. 5A through 5D illustrate exemplary forms of the present device shown positioned adjacent various exemplary anatomical structures or lesions such as an appendage (FIG. 5A), a patent foramen ovale (FIG. 5B), a coronary lesion (FIG. 5C), and a cranial-facial sinus (FIG. 5D).

FIG. 6 is an enlarged elevational view of a still further exemplary expandable component shown in the deployed or expanded state and equipped with a central lumen to permit the flow of blood through the expandable component.

FIG. 7 is a schematic view of a system employing a device for delineating a structure assembled in accordance with the teachings of the present invention and employing an imaging device.

### DETAILED DESCRIPTION

Although the following text sets forth a detailed description of numerous different embodiments, it should be understood that the legal scope of the invention is defined by the words of the claims set forth at the end of this document. The detailed description is to be construed as exemplary only and does not describe every possible embodiment since describing every possible embodiment would be impractical, if not impossible. Numerous alternative embodiments could be implemented, using either current technology or technology developed after the filing date of this patent, which would still fall within the scope of the claims.

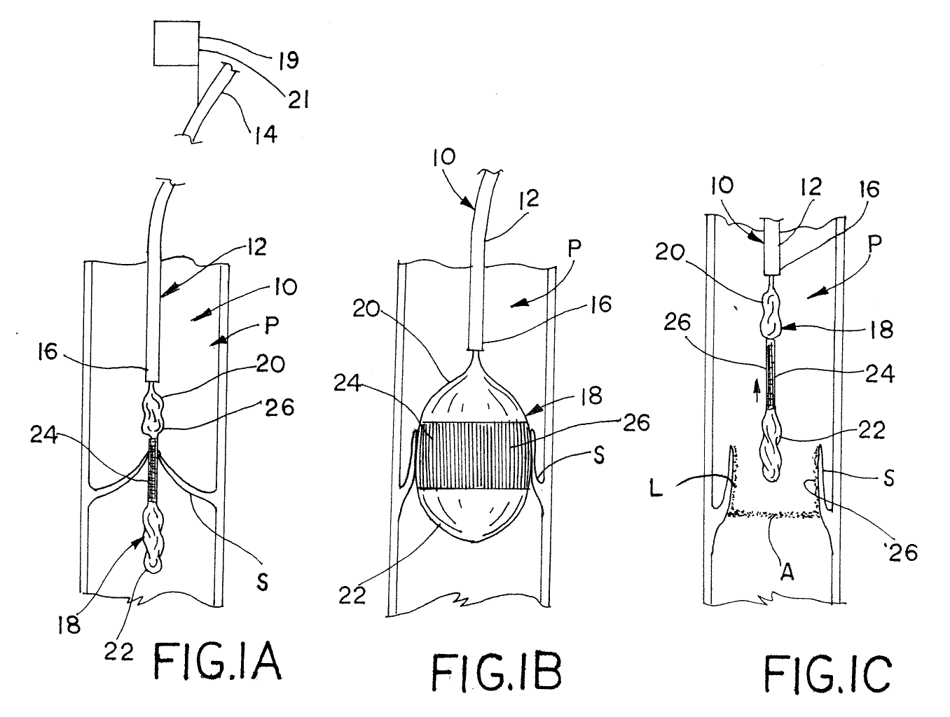

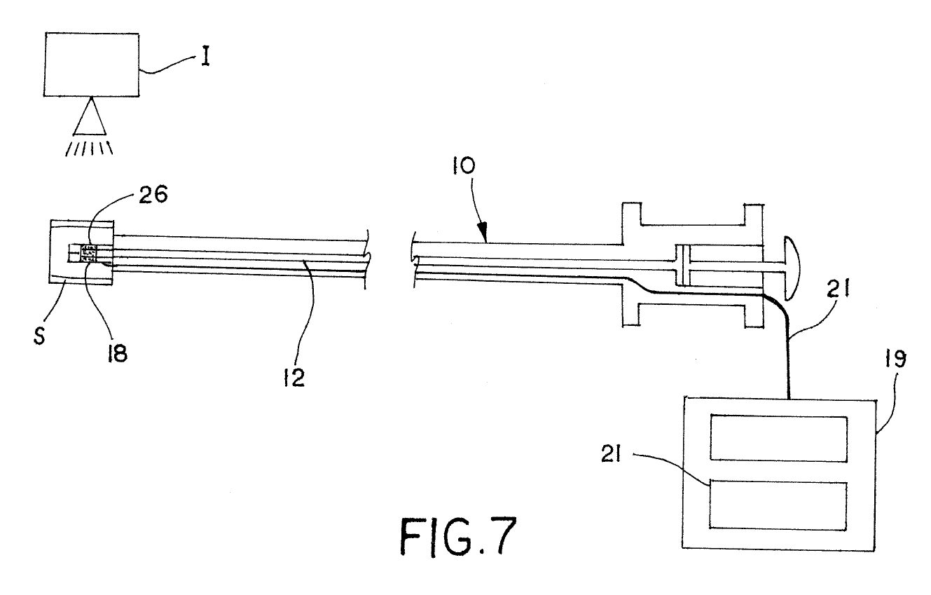

Referring now to FIGS. 1A through 1C of the drawings, a device for delineating a structure S is shown and is referred to by the reference numeral 10. The structure S may be, for example, a cardiovascular structure, a coronary lesion, or any other anatomical structure or lesion. The device 10 includes a catheter 12 having a proximal end 14 and a distal end 16, with the catheter 12 sized for insertion through a pathway P to a position adjacent the structure S. In the example of FIGS. 1A through 1C, the pathway P is coronary artery such as the aorta, and the structure S is the aortic valve. The device 10 includes an expandable component 18 which, in the example shown, protrudes from the distal end 16 of the catheter 12. The expandable component 18 includes a proximal portion or segment 20, a distal portion or segment 22, and an intermediate portion or segment 24. The expandable component 18 is, in the example of FIGS. 1A through 1C, suitably coupled to a source 19 housing an expansion medium 21 which may be, for example, air or any suitable gas, or any suitable liquid. Using the expansion medium 21, each of the proximal, distal, and intermediate segments 20, 22 and 24 are shiftable between a collapsed state as shown in FIGS. 1A and 1C, and an expanded state as shown in FIG. 1B. As shown, the expandable component 18 is sized to position the proximal and distal segments 20 and 22 on opposite sides of the structure S, such that the intermediate segment 24 is positioned generally adjacent the structure S. A marking medium 26 is carried by the intermediate segment 24 of the expandable component 18. As will be outlined in greater detail below, the marking medium 26 is detectable using a selected imaging device I (shown schematically in FIG. 7). As will be discussed in greater detail below, a user (not shown) positions the device 10 with the expandable component 18 in the desired positioned adjacent the structure S while the expandable component 18 is in the collapsed state as shown in FIG. 1A. Upon placement at the desired position, the expandable component is expanded using the expansion medium 21 from the source 19, so that the expandable component 18 expands to the deployed or expanded position shown in FIG. 1B. As shown in FIG. 1B, with the expandable component 18 in the expanded position, and with the expandable component 18 positioned adjacent the structure S as shown, the marking medium 26 is transferred from the expandable component 18 to the structure S. As shown in FIG. 1C, upon returning all three segments 20, 22 and 24 of the expandable component 18 to their original collapsed states, it can be seen that the marking medium 26 has been transferred to the structure S. In the specific example shown, the marking medium 26 has been transferred to one or all of the leaflets L of the aortic valve, and/or to the annulus A of the aortic valve. Consequently, using a suitable imaging device such as the imaging device I illustrated schematically in FIG. 7, the position and/or the orientation of the structure S can now be readily discerned.

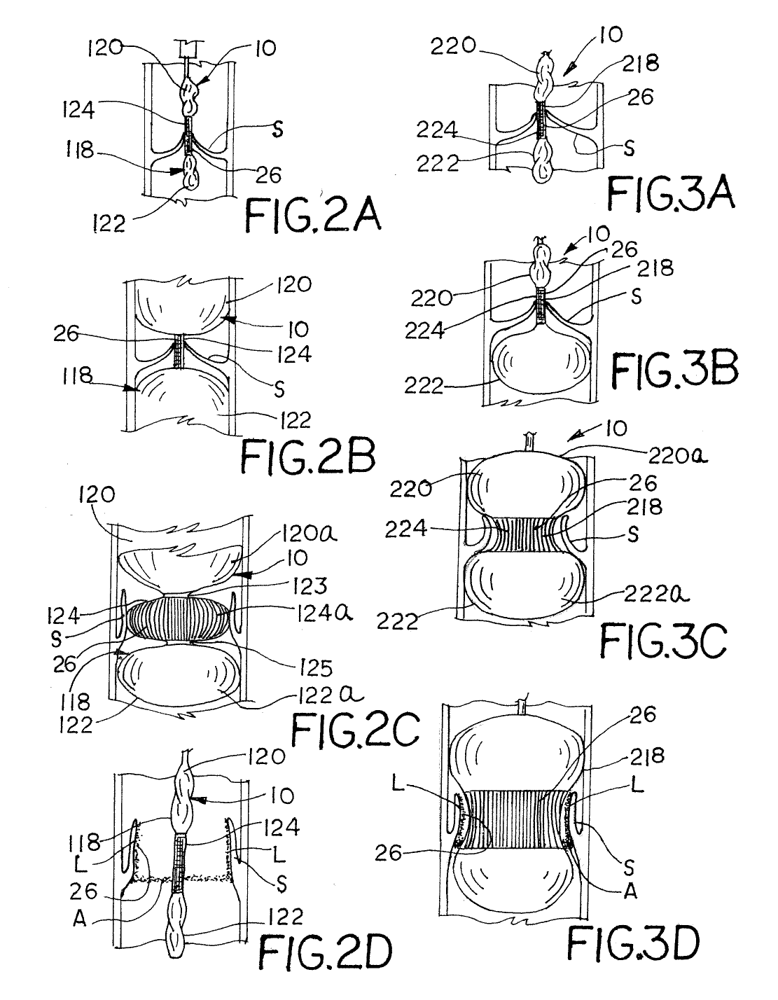

Referring now to FIGS. 2A through 2D, an alternative form of the expandable component 18 is shown and is referred to by the reference numeral 118. Except for the differences between the expandable component 118 shown and the expandable component 18 discussed above, the remaining components of the device 10 may be the same or substantially similar and therefore, for the sake of brevity, need not be discussed further herein. The expandable component 118 includes proximal, distal, and intermediate segments 120, 122 and 124, respectively. The expandable component 118 is suitably connected to the source 19 of the expansion medium 21 as discussed above. In the example shown, the expandable component 118 is arranged for differential or independent expansion. More specifically, the proximal and distal segments 120 and 122 may be inflated or expanded in one step as shown in FIG. 2B, while the intermediate segment 124 having the attached marking medium 26 remains in the collapsed state. Consequently, with the intermediate segment 124 properly positioned adjacent the structure S, the intermediate segment 124 can then be expanded independently from the proximal segment 120 and the distal segment 122 as shown in FIG. 2C, such that the marking medium 26 is transferred to the structure S in the manner described above. In the example shown in FIGS. 2A through 2D, the proximal, distal, and intermediate segments 120, 122 and 124 take the form of expanded or protruding sections or bulbs 120a, 122a and 124a, (shown in FIG. 2C) separated by narrowed sections 123 and 125 (also shown in FIG. 2C). Subsequently, as with the foregoing example, the expandable component 118 is returned to the collapsed state for withdrawal. As with the foregoing example, the leaflets L and/or the annulus A have been marked by the marking medium 26 and are now discernible using the selected imaging technique.

Referring now to FIGS. 3A through 3D, another alternative form of the expandable component is shown and is referred to by the reference numeral 218. Except for the differences between the expandable component 218 shown and the above-described expandable components, the remaining components of the device 10 may be the same or substantially similar and therefore, for the sake of brevity, again need not be discussed further herein. The expandable component 218 includes proximal, distal, and intermediate segments 220, 222 and 224, respectively. The expandable component 218 is suitably connected to the source 19 of the expansion medium 21 as discussed above. In the example shown, the expandable component 218 is again arranged for differential or independent expansion. More specifically, the proximal and distal segments 220 and 222 may be inflated or expanded independently of one another. For example, after positioning the device 10 adjacent the structure S, the distal portion 222 may be expanded first as shown in FIG. 3B, while the proximal and intermediate segments 220 and 224, respectively, remain in the collapsed state. This configuration may be helpful in proper positioning of the device 10 adjacent the structure S. Consequently, with the intermediate segment 224 having the marking medium 26 properly positioned adjacent the structure S and the distal segment 222 generally abutting a distal side of the structure S, the proximal segment 220 can then be expanded as shown in FIG. 3C, with the intermediate segment 224 remaining in the collapsed state or at least in a narrowed state. Subsequently, with the device positioned properly as shown in FIG. 3C, the intermediate segment 224 can then be fully expanded independently as shown in FIG. 3D, such that the marking medium 26 is transferred to the structure S in the manner described above. In the example shown in FIGS. 2A through 2D, the proximal and distal segments 220 and 222 take the form of expanded or protruding sections or bulbs 220a and 222a, separated by the narrowed intermediate segment 224, until the intermediate segment is fully expanded as shown in FIG. 3D. Subsequently, as with the foregoing example, the expandable component 218 is returned to the collapsed state for withdrawal. As with the foregoing example, the leaflets L and/or the annulus A have been marked by the marking medium and are now discernible using the selected imaging technique.

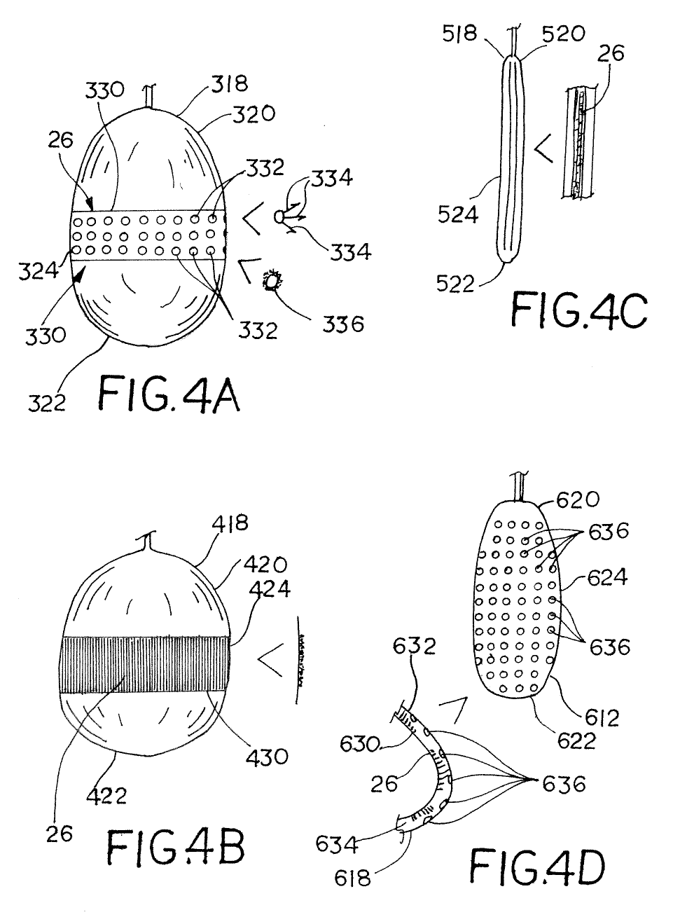

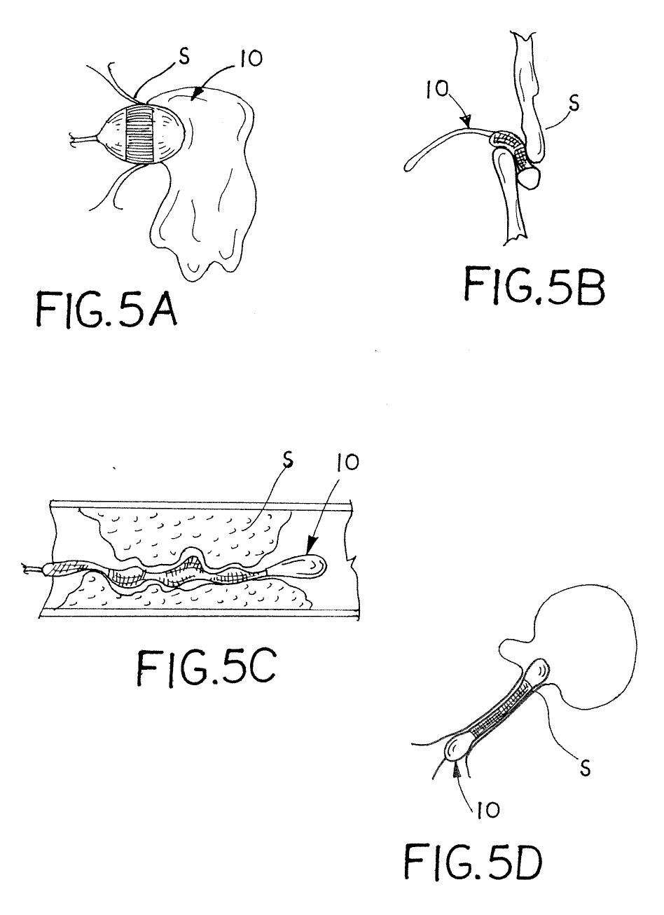

Referring now to FIG. 4A, another exemplary expandable component 318 is shown and is suitable for use with the above-described device 10. The expandable component 318 includes proximal, distal and intermediate segments 320, 322 and 324 and may incorporate one or more additional features of the above-described examples. Specifically, the expandable component 318 may be arranged for expansion all at once as shown in FIGS. 1A through 1C, or for independent and/or differential expansion of the various segments as shown in FIGS. 2A through 2D and FIGS. 3A through 3D. In the example of FIG. 4A, the marking medium 26 is arranged in a band or pattern 330 consisting of a plurality of individual or discrete points or dots 332. Each of the dots 332 includes the marking medium 26. Each of the dots 332 may include a plurality of barbs 334, or each of the dots 332 may be treated with an adhesive 336. The barbs 334 may take a number of possible forms which allow mechanical securement of the dots 332 to the selected area of tissue at the structure S (not shown). Similarly, the adhesive 336 may take the form of an adhesive, a chemical bonding agent, or any other suitable material that permits the dots 332 to be secured to the selected area of tissue at the structure S. The dots are releasable or transferable to the surrounding tissue upon expansion of the expandable component 318 to be expanded positioned shown in FIG. 4A.

Referring now to FIG. 4B, a still further another exemplary expandable component 418 is shown and is suitable for use with the above-described device 10, and again includes proximal, distal, and intermediate segments 420, 422 and 424. The expandable component 418 may incorporate one or more features of the above-described examples, and again may be arranged for independent and/or differential expansion of the various segments as shown in the Figures and as described above. In the example of FIG. 4B, the marking medium 26 is arranged in a band 430 carried by the intermediate segment and generally surrounding the expandable component 418. The marking medium 26 is treated with a bonding agent 436 which may take the form of, for example, an adhesive, a polymer, other suitable chemical bonding agents, or any combination thereof, that permit the marking medium 26 to be secured to the selected area of tissue at the structure S. The marking medium 26 is again transferable to the surrounding tissue upon expansion of the expandable component 418 to be expanded positioned shown in the Figure.

Referring now to FIG. 4C, yet another exemplary expandable component 518 is shown and is suitable for use with the above-described device 10, and again includes proximal, distal and intermediate segments 520, 522 and 524. The expandable component 518 may incorporate one or more features of the above-described examples, and again may be arranged for independent and/or differential expansion of the various segments as shown in the Figures and as described above. In the example of FIG. 4C, the marking medium 26 is arranged in one or more lines 530 carried by at least the intermediate segment, such that the lines 530 are distributed circumferentially around the expandable component 518. Each of the lines 530 is generally defined by a fold line 532 formed in the expandable component 518 which, in the example of FIG. 4C, takes the form of an expandable balloon. Each of the fold lines 532 includes a quantity of the marking medium 26, with the marking medium 26 remaining enclosed in the fold lines 532 when the balloon is in the collapsed state. When the balloons expanded to the expanded state, the fold lines 532 open up as shown in the right-hand portion of FIG. 4C, bus enabling a marking medium 26 to be released to the surrounding tissue.

Referring now to FIG. 4D, a further exemplary expandable component 618 is shown and is suitable for use with the above-described device 10. The expandable component 618 may incorporate one or more features of the above-described examples, and again may be arranged for independent and/or differential expansion of the various segments 620, 622 and 624 as shown in the Figures and as described above. In the example of FIG. 4D, the expandable component includes an inner balloon 630 and an outer balloon 632, defining a space 634 between the inner and outer balloons. The inner and outer balloons at 630 and 632 may be independently expandable. The outer balloon 632 includes a plurality of apertures or pores 636. A marking medium 26 is distributed to surrounding tissue at the structure S by expanding the outer balloon 632 using an expansion medium after a device has been positioned in the proper location. Subsequently, the inner balloon is expanded using any suitable means, such as the same expansion medium or a different expansion medium, which causes the marking medium 26 contained in the space 634 to be ejected through the pores 636.

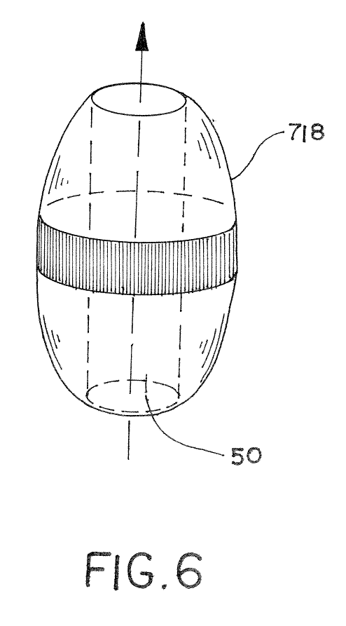

Referring now to FIG. 6, any one of the foregoing expandable components may be provided with a central lumen 50. Here, an expandable component 718 is shown, which may be the same or similar to any of the foregoing expandable components. Upon inflation or expansion of the relevant expandable component, the central lumen 50 allows for blood flow through the device during use.

Referring now to the schematic of FIG. 7, the expansion medium 19 is connected to the expandable component 19 via a suitable link or conduit 21. For any of the expandable components discussed above having differential expansion capabilities, the link or conduit 21 may have multiple lumens or pathways, or the link 21 may be formed of a number of independent conduits. The expandable component is shown positioned at the structure S (illustrated only schematically in FIG. 7). The system shown therein includes an imaging device I, which may be any suitable imaging device. As explained herein, the marking medium 26 is visible using the imaging device.

In accordance with one or more aspects of the preferred forms disclosed herein, a percutaneous balloon with a marking medium 26 forming deployable markings may be used to delineate the structure S in the form of an anatomical structural defect or a vascular lesion, or any other structure to be treated. The resulting markings placed on the structure by the marking medium 26 may be visualized during subsequent fluoroscopic placement of a permanent medical device. The markings may be a partial or total coating of a balloon or other expandable component with, for example, radiographic material, such as iodinated contrast. The radiographic material may be mixed with a hydrophobic or hydrophilic polymer, or a compound with both of these properties, to enable fixation of the material against the target lesion (e.g., vascular endothelium, cardiac valve, left ventricular outflow tract, etc.). The marking material preferably has properties that allow adherence for a certain amount of time, during which the operator places a permanent medical device. Subsequently, a marking medium preferably dissolves into the vascular blood pool. The marking material may be contained in any one of the exemplary delivery vehicles outlined herein, such as beads or spheres, that may be contained within, for example, balloon folds, and that become adherent with the surrounding tissue upon expansion of the expandable component such as a balloon. Additionally, the marking material may consist of longitudinal or circumferential bands or other patterns on the balloon that take on the shape of the structural defect.

In accordance with the disclosed example, one exemplary intent is to transfer a marking substance to a tissue/structure of interest, from any one of the expandable components outlined herein containing a marking medium.

It is presently contemplated that some applications for the device described herein may include the following:

A) During percutaneous valve replacement, it is desirable that the valvular annulus be seen well during fluoroscopy. Balloon inflation with the radiographic marking balloon, which may be used for valvuloplasty, will delineate the outflow (or inflow) area, the diseased valve, and the valve annulus. This delineation will assist in more accurate and more precise percutaneous valve placement.

B) During treatment of pulmonary vein stenosis, the ostia and proximal portion of the pulmonary veins can be tortuous. The marking balloon will delineate the lesion to allow more accurate angioplasty and stenting of the stenosis.

C) For patients with congenital heart disease defects, the marking balloon can delineate rims before closure (including, for example, atrial septal defect, patent foramen ovale, ventricular septal defect), the ostium of a patent ductus arteriosus, and the precise location of coarctation in the aorta.

E) For patients undergoing percutaneous closure of an atrial appendage, the marking balloon will delineate the irregular orifice of the atrial appendage prior to placement of a Watchman device.

F) Peri-prosthetic leaks typically are serpiginous and not easily seen with echocardiography or fluoroscopy. The marking balloon will provide a better outline of the shape, size, and orientation of these defects to enable better device selection and more accurate or precise placement.

G) The device may be used for better placement of arterial stents and/or for better treatment of coronary artery lesions and plaques, bifurcated vessels, and/or ostial stent placement. All of these locations, as well as other locations and other anatomical structures, which may require stenting, all could benefit from a balloon-marking system as outlined herein to outline/mark the area for stent placement.

H) The device may be used in sinus cavities, such as during balloon sinuoplasty, as in such uses it may be advantageous to mark the lumen of the sinus to identify the area treated and the amount of patency that was generated both during the procedure and at subsequent follow up visits.

In further accordance with one or more of the disclosed examples, the expandable component could be a balloon (as outlined in many of the preferred forms discussed herein) or any other expandable, compliant material, such as a memory metal (e.g. Nitinol), or an expandable foam, or any other suitable expandable structure.

The marking substance forming the marking medium could be an agent that is visible by x-ray (heavy metals, iodine, barium, etc.) or by ultrasound (i.e. echogenic substances such as air, foam, particles formed from biopolymers such as Alignate and chitin, etc. See, for example, any one of the following patents or patent publications: W002078611, U.S. Pat. No. 5,921,933, published U.S. patent application 20090028797, W004006964, for examples of echogenic coatings and agents) or by MRI (gadolinium or chromium based agents). See U.S. Pat. Nos. 4,639,565 or 7,524,483 for examples of MRI contrast agents.

In further accordance with a preferred form, the marking substance could be combined with a therapeutic substance to combine imaging and therapy. Examples of therapeutic drugs which could be combined with the marking substance include taxol, rapamycin (and analogs), steroid, anti-inflammatory agents, etc.. Examples of balloons being used to deliver a therapeutic agent include U.S. Pat. Nos. 6,939,320, 6,146,358, and 7,179,251, and published application W009051614.

The marking substance can be applied and held onto the balloon by physical means, such as by keeping substance within a deflated balloon fold, by chemical means, such as by adhesives, bonding agents, or other suitable it here it's or means. See generally U.S. patent publications 200825510, 2006020243, 2008009746, and U.S. Pat. No. 5,102,402 for examples of coatings and physical means. The marking substance could be combined with a substance to promote differential adherence to a tissue of interest (e.g. endothelial cells, valve leaflets, nerve tissue, epithelial cells, etc.). Examples of such substances include: Endothelial cells, see U.S. published patent applications 2006/0024232, 2005/0063904, 2005/0207974 lectins/integrins (tissue specific) 2006/025158, and 2006/0147380. The marking device could include a means to facilitate deployment of the marking substance onto tissues or into cells (e.g. energy—RF or ultrasound, magnetic force, chemical bonding, thermal energy, fluid pressure, etc.)

The marking device could be in the deployed state during an acute procedure (e.g. valvuloplasty) for any length of time (e.g. 30 seconds to one hour). The marking substance could be designed to mark the tissue/structure for any length of time (30 seconds to one hour). Alternatively, the marking device could be used to mark a tissue/structure long term (e.g. mark an area during a procedure and have the marking substance mark the tissue/structure for days or weeks to allow the clinician to monitor the structure non-invasively over multiple visits/longterm).

A marking device may have an internal lumen that would allow the device to be in the deployed state for an extended period of time, while still allowing the lumen/structure to remain open/patent (e.g. allow blood flow) see FIG. 6.

The marking device may be differentially expandable to facilitate placement, localization, and ease of use (e.g. different areas of the device may expand prior to other areas) See FIG. 2 or FIG. 3

In the example of FIG. 1, the exemplary device is positioned with the balloon across a valve in the form of the aortic valve. The device is inflated causing the marking substance to contact the valve leaflets and annulus and mark those structures. Upon deflation of the balloon, the structures remain marked to facilitate subsequent device placement (e.g. synthetic valve). The deflated balloon may contain a marker element, in addition to the marking substance, to facilitate positioning of the balloon (see also FIG. 2A).

FIG. 2 shows differential expansion of the marking device. In FIG. 2A through 2D there is a “three-segment” balloon design where the proximal and distal segments inflate first to facilitate positioning. Once the correct position is established, the middle, third segment is inflated to deploy the marking substance. FIG. 2B shows an alternative expansion along the lines of an Inoue balloon (see weblink in email for inoue balloon function). In this instance the operator would inflate the balloon to get a distal lobe then pull back to catch the valve (correct positioning) and then inflate the balloon further to deploy the marking substance.

FIG. 4 shows various iterations of the marking device (balloon). FIGS. 4A through 4D show that the marking substance could be a drug/polymer/fluid or could be a device-like small disc that detaches from the balloon and adheres to tissues via barbs. The marking substance can be several small dots, one large surface area covering and in any geometry (vertical band, horizontal band, complete covering, etc.). FIG. 4B shows a horizontal band with a polymer/fluid marker coating. FIG. 4C shows a version with the marker substance within folds in the deflated balloon. FIG. 4 D shows a double walled balloon with inflation within the first, solid balloon and deployment of a marker/drug within the lumen formed by the first balloon wall and the second (pore containing) wall. There could be separate lines/ports for air injection and marker substance injection.

FIG. 6 shows a marking device with a central lumen to allow the device to expand and interact with tissue while allowing the vessel/structure to remain patent (e.g. arrow indicates blood flow in artery).

What is claimed:

1. A device for delineating a cardiovascular structure, comprising:
a catheter sized for insertion to a position adjacent the cardiovascular structure;
an expandable component operatively coupled to a distal end of the catheter, the expandable component including a proximal segment, a distal segment, and an intermediate segment, the expandable component sized to permit the proximal and distal segments to be disposed on opposite sides of the cardiovascular structure with the intermediate segment in a desired position adjacent the cardiovascular structure;
each of the proximal, distal and intermediate segments shiftable between a collapsed state and an expanded state, the intermediate segment shiftable independently of the proximal and distal segments;
a marking medium carried by a portion of the intermediate segment, the marking medium detectable using a selected imaging device; and
the marking medium arranged to be transferable from the intermediate segment to portions of the cardiovascular structure upon shifting the intermediate segment to the expanded position with the intermediate segment in the desired position.

2. The device of claim 1, wherein the expandable component comprises a memory metal.

3. The device of claim 1, wherein the expandable component comprises foam.

4. The device of claim 1, wherein the expandable component comprises a balloon.

5. The device of claim 1, wherein the marking medium comprises a substance detectable using x-ray imaging.

6. The device of claim 1, wherein the marking medium comprises a substance detectable using ultrasound.

7. The device of claim 1, wherein the marking medium comprises a substance detectable using at least one of:
(a) an optical coherence tomography device,
(b) an ultrasound device,
(c) a magnetic resonance imaging device,
(d) an x-ray imaging device,
(e) a fluorescence imaging device,
(f) a near-infrared fluorescence (NIRF) imaging device, and
(g) a camera.

8. The device of claim 1, wherein further comprising a therapeutic agent carried by the expandable component.

9. The device of claim 8, wherein the therapeutic agent and the marking medium are mixed.

10. The device of claim 1, wherein the marking medium is chemically adhered to the expandable component.

11. The device of claim 1, wherein the intermediate segment of the expandable component is foldable along fold lines when in an initial collapsed state, and wherein the marking medium is secured within the fold lines when the intermediate segment is in the initial collapsed state.

12. The device of claim 1, wherein the marking medium is combined with an adherent substance, the adherent substance arranged for differential adherence to different tissues.

13. The device of claim 1, wherein the marking medium comprises a means to facilitate deployment of the marking medium.

14. The device of claim 13, wherein the means is selected from a material responsive to at least one of FR energy, ultrasound, magnetic energy, chemical bonding, thermal energy, and fluid pressure.

15. The marking device of claim 1, wherein the marking medium is bioabsorbable.

16. A method for delineating an anatomical structure, comprising:
providing a catheter sized for insertion to a position adjacent the structure;
providing an expandable component operatively coupled to a distal end of the catheter, the expandable component including a proximal segment, a distal segment, and an intermediate segment;
positioning the expandable component to place the proximal and distal segments on opposite sides of the structure, with the intermediate segment in a desired position adjacent the cardiovascular structure;
equipping each of the proximal, distal and intermediate segments to be shiftable between a collapsed state and an expanded state;
equipping the intermediate segment to be shiftable independently of the proximal and distal segments;
providing at least the intermediate segment with a marking medium carried by a portion of the intermediate segment, the marking medium detectable using a selected imaging device;
positioning the device adjacent the anatomical structure;
expanding the segments to transfer the marking medium from the intermediate segment to portions of the anatomical structure.

17. The method of claim 16, including deflating the segments after transferring the marking medium to the structure.

18. The method of claim 17, including withdrawing the catheter and the expandable component after deflation, and using an imaging device to ascertain characteristics of the structure.

19. The method of claim 18, including using the characteristics to position a medical device.

20. The method of claim 16, including delivering a therapeutic agent through the expandable component.

21. The method of claim 20, including mixing the therapeutic agent and the marking medium.

22. The method of claim 16, including chemically adhering the marking medium to the expandable component.

23. The method of claim 16, including carrying the marking medium on the intermediate segment of the expandable component by placing the marking medium in fold lines in the expandable component.

24. The method of claim 16, including combining the marking medium with an adherent substance, the adherent substance arranged for differential adherence to different tissues.

25. The method of claim 16, including providing the marking medium with means to facilitate transfer of the marking medium to the structure, wherein the means is selected from a material responsive to at least one of FR energy, ultrasound, magnetic energy, chemical bonding, thermal energy, and fluid pressure.

26. The method of claim 16, including expanding the expandable segments to an intermediate state prior to expanding to the expanded state.

27. The method of claim 16, including using the selected imaging device in real-time during delineation of the anatomical structure.
